(19) United States
(12) Patent Application Publication
(10) Pub. No.: US 2024/0350534 A1
(43) Pub. Date: Oct. 24, 2024

(54) IODINE DELIVERY COMPOUNDS

(71) Applicant: IOCURE, INC., Lawrence, NY (US)

(72) Inventor: Mark Daniel Farb, Beit Shemesh (IL)

(21) Appl. No.: 18/260,940

(22) PCT Filed: Dec. 10, 2021
(86) PCT No.: PCT/US2021/072845

§ 371 Date: Jul. 11, 2023

(51) Int. Cl. A61K 33/18; A61K 31/775; A61K 47/68; A61K 47/69; A61P 31/06; A61P 35/00

(52) U.S. Cl. CPC A61K 33/18; A61K 31/775; A61K 47/6803; A61K 47/6913; A61P 31/06; A61P 35/00

(57) ABSTRACT

Disclosed herein are methods and compositions for manufacturing pharmaceutical compositions and treating and ameliorating pathological conditions, using carriers conjugated to iodine-containing compounds; and therapeutic mixtures including iodine-containing compounds

### FIELD

Disclosed herein are methods of manufacturing pharmaceutical compositions, and methods and compositions for treating and ameliorating pathological conditions, using conjugated iodine-containing compounds. Also disclosed are therapeutic mixtures including iodine-containing compounds.

### BACKGROUND

Iodine has much more therapeutic potential than as a skin disinfectant and chemical for affecting the thyroid. The inventor has recently explained how steamed iodine in solution can be delivered safely to the lungs. This enables the active ingredient, the I₂, that is present in small quantities in a solution of an iodine-containing compound, such as potassium iodide, to react with a pathogen and draw more I₂ out of the solution. Here is one example of how an equilibrium enables this to happen:

KI+I₂<-->KI₃.

As I₂ reacts with the pathogen, more is formed. Little I₂ is available to create toxicity for the host, human or animal.

Homann et al. and Reimer et al. describe use of a liposome hydrogel with polyvinyl-pyrrolidone iodine in the local treatment of burn wounds and as an anti-microbial eyedrop. The hydrogel is used to “provide the moist environment that promotes fast wound healing” and/or prolonging release of the active ingredient. The inventor is not aware of use of similar compositions internally, e.g., to target cells and intracellular pathogens with non-radioactive iodine and iodine-containing compounds.

### SUMMARY

We can extend the usefulness of iodine and iodine-containing compounds with a carrier or envelope to bring iodine to the location where it is needed. As examples, nanoparticles and antibody carriers, particularly those specific for a certain type of cell, can fulfill this role. Radioactive iodine has been used in thyroid disease. Their combination with iodine, in one embodiment non-radioactive, and in another embodiment radioactive iodine, is described herein. The advantage of being able to deliver non-radioactive iodine means that one can ablate sensitive cells or organisms without the risk of radioactivity. An example is breast cancer cells, which are sensitive to iodine. In another embodiment the described methods and compositions increase specificity of radioactive iodine-containing compositions. In various embodiments, the disclosed methods and compositions include combining, conjugating, encircling, enclosing, attaching, or in any way coupling an iodine atom, molecule, salt, or substance containing or releasing iodine to an organic compound, in one embodiment a nanoparticle, in another embodiment an immunoglobulin, and in another embodiment a combination of any of the above.

In other embodiments, the described methods and compositions enable specific delivery of iodine to target tissues, cells, or pathogens without systemic toxicity. If the nanoparticles bind with membrane receptors properly, they can then enter the cell, which internalizes the drug that is being carried.

One way of using nanoparticles to reach the desired cell is active targeting. They usually target cells by ligand-receptor interaction or antibody-antigen recognition. Usually, an active targeting entity has 3 components: (1) the active drug, in this case, we claim, iodine or an iodine containing compound; (2) a targeting moiety or penetration enhancer; and (3) a carrier. The active drug can be entrapped, covalently bound, encapsulated, or adsorbed, among other embodiments of coupling.

### DETAILED DESCRIPTION

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

Aspects of the invention relate to methods of manufacturing pharmaceutical compositions, and methods and compositions for treating and ameliorating pathological conditions, using iodine-containing compounds conjugated to a carrier. Also disclosed are therapeutic mixtures including iodine-containing compounds.

In some embodiments, there is provided a pharmaceutical composition, comprising an iodine-containing compound conjugated (or in other embodiments coupled) to a carrier. In certain embodiments, the carrier is a nanoparticle. In other embodiments, the carrier is a liposome. In still other embodiments, the carrier is selected from a liposome, a polymeric micelle, a nanosphere, a nanocapsule, and a nanotube. In more specific embodiments, the iodine-containing compound is disposed within the nanoparticle; or in other embodiments the liposome, polymeric micelle, nanosphere, nanocapsule, or nanotube. Alternatively, or in addition, the nanoparticles—or, in other embodiments, the liposomes, polymeric micelles, nanospheres, nanocapsules, or nanotubes—display a targeting moiety. Use of nanoparticles is known in the art (e.g., Sutradhar et al., and the references cited therein). The iodine or iodine-containing compound contained in any of the pharmaceutical compositions mentioned herein may be non-radioactive, or, in other embodiment, radioactive. It may be any embodiment of iodine or iodine-containing compounds mentioned herein, each of which represents a separate embodiment.

In certain embodiments, the described pharmaceutical compositions are indicated for internal use, defined as non-topical use.

Without wishing to be bound by theory, in the case of liposomes and other carriers with a hydrophilic exterior, the exterior can aid in delivery of the iodine, and a hydrophobic interior holds the iodine compound until it reaches the cell and is phagocytosed. Encapsulation of iodine-containing compounds in liposomes is known in the art, and is described, for example, in Reimer et al., Homann et al., and the references cited therein.

The term “conjugated”, in various embodiments, may refer to combining, conjugating, encircling, enclosing, attaching, or in any way coupling the iodine-containing compound to the carrier, including via non-covalent bonds, or, in other embodiments, covalent bond(s).

In other embodiments, there is provided a method of producing a pharmaceutical composition, comprising encapsulating an iodine-containing compound within a nanoparticle.

In still other embodiments, the carrier is an immunoglobulin, an envelope (e.g., a lipid envelope), a dendrimer, or a protein.

### Iodine, Iodine-Releasing Ionic Compounds, and Solutions Comprising Same

The iodine in the described iodine-containing compound is, in some embodiments, non-radioactive. In other embodiments, the iodine is radioactive. Each embodiment represents a separate embodiment.

The term “iodine” is used herein to refer to the element itself, e.g., in its common molecular form “I subscript 2”, or in another embodiment, an iodine atom. Iodine-releasing compounds encompass iodine-releasing salts, such as hypoiodous acid; and ionic molecules containing and releasing iodine. Solutions containing iodine and iodine-releasing compounds are also encompassed in the described methods, compositions, uses, and articles of manufacture. In certain embodiments, the described compounds are able to generate bioavailable iodine (I sub 2). In certain embodiments, iodine is present in the described compositions in a therapeutically effective amount, that is, an amount having anti-microbial or anti-cancer activity. In other embodiments, iodine is present in any of the amounts or ranges mentioned herein. Alternatively, or in addition, two or more separate iodine-containing compounds are present. In certain embodiments, the two or more iodine-containing compounds form an equilibrium. Non-limiting examples of such combinations of iodine-containing compounds are molecular iodine combined with an iodide compound, e.g., sodium iodide, potassium iodide, or hydrogen iodide.

The described iodine-releasing compounds (of any method, composition, use, or article of manufacture mentioned herein) include, in certain embodiments, compounds that release free elemental iodine or another active iodine compound in solution. Those skilled in the art will also appreciate, considering the present disclosure, that the particular active iodine species is not critical for reducing to practice the described methods and compositions.

In other embodiments, the described iodine-releasing compound (of any method, composition, use, or article of manufacture mentioned herein) is hypoiodous acid (HOI), povidone iodine (2-Pyrrolidinone, 1-ethenyl-homopolymer), an organic or inorganic iodine carrier, or an iodine salt, such as potassium iodide, each of which represents a separate embodiment. In certain embodiments, the iodine salt is potassium iodide, sodium iodide, or a mixture thereof. In still other embodiments, uncomplexed molecular iodine (I sub 2) is the active ingredient.

In certain embodiments, povidone iodine is formulated in a hydrogel composition, e.g., containing gelatin, wherein the rate of release corresponds to the hydrogel composition, decreasing with more gelatin, as is known in the art (Bigliardi et al.). Therefore, adding a high enough percentage of gelatin to enable the povidone in an envelope to reach its target without releasing iodine will selectively release iodine at the desired destination. In certain embodiments, the percentage, or in other embodiments, the concentration, of gelatin is adjusted to meet the targeting requirements of the target tissue.

It will be appreciated by those skilled in the art, in light of the present disclosure, that the term “active iodine compound(s)” refers to iodine-containing compounds with significant antibiotic or anti-tumor activity at physiological concentrations.

In yet other embodiments, the described pharmaceutical composition further comprises a penetration-enhancing moiety. In certain embodiments, such a moiety enhances transport of iodide into a target cell or, in other embodiments, a free virion. In more specific embodiments, the target cell is an infected cell; or, in other embodiments, a cancer cell.

### Ion Channels

In certain embodiments, the penetration-enhancing moiety is an ion channel. In another embodiment, the penetration-enhancing moiety interacts with an ion channel, e.g., by increasing its activity. In certain embodiments, the ion channel is a naturally occurring channel, non-limiting examples of which are sodium/iodide cotransporter (NIS; Son et al.; UniProt Accession No. Q92911); Volume-regulated anion channel (UniProt Accession No. Q8IWT6 describes one of the subunits); Pendrin (UniProt Accession No. O43511); Anoctamin-1 (UniProt Accession No. Q5XXA6); and NADPH oxidase 3 (UniProt Accession No. Q9HBY0). In other embodiments, the ion channel is an artificial channel, non-limiting examples of which are PB-1A (PhysOrg 2017) and others mentioned herein. In preferred embodiments, the channel has significant activity for iodide atoms.

A non-limiting example of the described ion channel is NIS, a naturally occurring glycoprotein that enhances the transport of sodium and iodide into the cell (Son et al. and Micali et al.). Attaching this to other particles that specifically target an infected or cancerous cell could enable more transport in selected cells. In another embodiment, when combined with other means of delivering iodine compounds simultaneously, the delivery of iodine to cell targets can be enhanced. In certain embodiments, non-radioactive NIS is implanted in the target cell membrane through attached particles.

A non-limiting example of a synthetic ion channel that mimics the function of NIS (Liu et al., and Benke et al.), as well as those described in Wu et al., and the references cited therein; and pore-forming helically folded polyhydrazides, e.g., foldamer-based polyhydrazides, non-limiting examples of which are described in Roy et al. In certain embodiments, the described compositions enable delivery of non-radioactive—or, in other embodiments, radioactive iodine—to selected tissues without systemic toxicity.

In still other embodiments, there is provided use of a described pharmaceutical composition for treating a subject infected with an intracellular pathogen. In yet other embodiments, there is provided a method for treating a subject infected with an intracellular pathogen, comprising administering a described pharmaceutical composition to the subject. In more specific embodiments, the described carrier comprises a targeting moiety, for example a moiety that binds a surface marker of the intracellular pathogen. In certain embodiments, the targeting moiety is a receptor for the surface marker. The surface marker of the intracellular pathogen may be, in various embodiments, a protein, a lipid, a carbohydrate, or a glycoprotein. In various embodiments, the subject is a human, or, in other embodiments, an animal.

Reference herein to an intracellular pathogen, refers, in some embodiments, to a pathogenic microbe that ordinarily lives and replicates inside a host cell. In certain embodiments, the pathogen is a gram-negative bacterium; in other embodiments, the pathogen is a gram-positive bacterium. Non-limiting examples of pathogenic bacteria are such as Bartonella henselae, Francisella tularensis, Listeria monocytogenes, Salmonella Typhi, Brucella Legionella, Mycobacteria (non-limiting examples of which are Mycobacterium tuberculosis, Mycobacterium bovis, Mycobacterium leprae, Mycobacterium ulcerans, and Mycobacterium marinum), Nocardia (non-limiting examples of which are Nocardia brasiliensis, Nocardia cyriacigeorgica, Nocardia farcinica, Nocardia nova, and Nocardia asteroides), Neisseria (non-limiting examples of which are N. meningitidis and N. gonorrhea), Rhodococcus equi, Yersinia (non-limiting examples of which are Yersinia pestis, Yersinia pseudotuberculosis, and Yersinia enterocolitica), Staphylococcus aureus, Chlamydia (non-limiting examples of which are Ch. trachomatis, Ch. pneumoniae, Ch. abortus, and Ch. psittaci), Coxiella (e.g., C. burnetii), Histoplasma capsulatum, and Cryptococcus neoformans. Surface antibodies of infected cells are known in the art, non-limiting examples of which are tuberculosis (TB) antigens such as 19 kD lipoprotein (Noss EH, et al.), and Rv0232 and Rv1115 (Li et al.)

In other embodiments, the pathogen is a virus. In more specific embodiments, the virus may be COVID-19. In other embodiments, the coronavirus described herein is human and bat severe acute respiratory syndrome coronavirus (SARS-COV) of the type severe acute respiratory syndrome-related coronavirus, e.g. SARS-COV-1 and SARS-COV-2.

In other embodiments, the described virus is any virus in the family Coronaviridae, e.g. comprising Cornidovirineae (Orthocoronavirinae) and Letovirinae. In other embodiments, the described virus is any virus in the suborder Cornidovirineae, e.g., comprising Alphacoronaviruses, Betacoronaviruses, Gammacoronaviruses, and Deltacoronaviruses

In other embodiments, the described virus is any virus in the order Nidovirales, e.g., comprising Cornidovirineae, Tornidovirineae, Mesnidovirineae, Ronidovirineae, Nanidovirineae, and Arnidovirineae.

In other embodiments, the described virus is any virus in the realm Riboviria. Taxonomy of coronaviruses is known to those skilled in the art, and is described, e.g., in Siddell, SG et al. 2019; Ziebuhr, J et al., 2017; Ziebuhr. J. et al., 2019; and Gorbalenya, S et al. (2020).

In certain embodiments, the treated virus is SARS-COV-2, e.g. a virus having a sequence at least 96%, or in other embodiments, at least 91%, at least 92%, at least 93%, at least 94%, at least 95%, at least 96%, at least 97%, at least 98%, or at least 99% homologous to at least 1 sequence selected from the nucleotide sequences set forth in GenBank Accession Nos. NC_045512.2, MT126808, MT123290, MT093571, MT066176, MT263074, MT276331, MT233523, MT066156, and LC528233.

In other embodiments, the treated coronavirus has a sequence at least 96%, or in other embodiments, at least 91%, at least 92%, at least 93%, at least 94%, at least 95%, at least 96%, at least 97%, at least 98%, or at least 99% homologous to at least 1 sequence selected from the nucleotide sequences set forth in GenBank Accession Nos. NC_004718.3, AY274119.3, GU553363.1, DQ182595.1, AY297028.1, and AY515512.1.

In other embodiments, the treated virus is related to a bat coronavirus, e.g. related to GenBank Accession No. DQ022305.

In still other embodiments, the virus is a coronavirus, an influenza virus, a respiratory syncytial virus, a vaccinia virus, a bovine viral diarrhea virus, a polyomavirus SV40, an adenovirus, a mumps virus, a rotavirus, a coxsackievirus, a rhinovirus, a herpes simplex virus, rubella, measles, or a poliovirus, each of which represents a separate embodiment. In other embodiments, the pathogen is another viral pathogen, each of which represents a separate embodiment.

In other embodiments, the virus is a lipid-enveloped virus; while in other embodiments, the virus is not lipid enveloped. Alternatively or in addition, the virus expresses a haemagglutinin, a neuraminidase, or both.

In certain embodiments, without wishing to be limited by theory, the described methods and compositions exert an effect by facilitating penetration of the cell membrane and access of the reactive iodine species to the pathogens therewithin. Iodine is effective against these organisms when outside the cell. Using a carrier can enable penetration into the cell and, at minimum, prevent the release of pathogens from infected cells. In the cases above, iodine has been shown to be a powerful antibiotic in vitro, and should be so in vivo if targeted to the needed location.

In yet other embodiments, there is provided use of a described pharmaceutical composition for treating a neoplasm. In other embodiments, there is a provided a method for treating a neoplasm in a subject in need thereof, comprising administering a described pharmaceutical composition to the subject. In more specific embodiments, the pharmaceutical composition further comprises a chemotherapeutic moiety. In certain embodiments, the neoplasm is malignant. In other embodiments, the neoplasm is benign. Alternatively or in addition, the pharmaceutical composition may be for internal use. In various embodiments, the subject is a human, or, in other embodiments, an animal.

In yet other embodiments, there is provided use of a described pharmaceutical composition for treating a tumor. In other embodiments, there is a provided a method for treating a tumor in a subject in need thereof, comprising administering a described pharmaceutical composition to the subject. In more specific embodiments, the pharmaceutical composition further comprises a chemotherapeutic moiety. In certain embodiments, the pharmaceutical composition is for internal use.

In yet other embodiments, there is provided use of a described pharmaceutical composition for treating a cancer. In other embodiments, there is a provided a method for treating a cancer in a subject in need thereof, comprising administering a described pharmaceutical composition to the subject. In more specific embodiments, the pharmaceutical composition further comprises a chemotherapeutic moiety.

In certain embodiments, the cancer treated by the methods or compositions mentioned herein is a breast cancer; in more specific embodiments, a breast carcinoma; in more specific embodiments, a breast ductal carcinoma; in more specific embodiments, an invasive breast ductal carcinoma. In other embodiments, the breast cancer is a mammary adenocarcinoma. In other embodiments, the breast cancer is metastatic mammary adenocarcinoma. Those skilled in the art will appreciate in light of the present disclosure that breast cancer tumors are sensitive to iodine (Oncology Times 2016), which states “Data from these studies indicated that molecular iodine has potent inhibitory effects on cell growth in . . . breast cancer . . . ”. In more specific embodiments, liposomes comprising an iodine-releasing compound are used to target breast cancer cells (Goldman et al 2017). In certain embodiments, the liposomes further comprise antibodies to cancer antigens (e.g., breast cancer antigens), non-limiting examples of which are human epidermal growth factor receptor 2 (HER2); Mucin 1 (MUC-1); Carcinoembryonic antigen (CEA); and Tn, TF, and sialyl-Tn (STn) antigens.

In certain embodiments, the cancer is selected from: acute lymphoblastic leukemia, adrenocortical carcinoma, AIDS-related lymphoma, anal cancer, appendix cancer, astrocytoma (childhood cerebellar or cerebral), basal cell carcinoma, bile duct cancer, bladder cancer, bone cancer, brainstem glioma, brain tumor (cerebellar astrocytoma, cerebral astrocytoma/malignant glioma, ependymoma, medulloblastoma, supratentorial primitive neuroectodermal tumor, visual pathway and hypothalamic gliomas), bronchial adenoma, carcinoid tumor of the lung, gastric carcinoid, other carcinoid tumors (e.g. childhood), Burkitt lymphoma, carcinoma of unknown primary, central nervous system lymphoma (e.g. primary), cerebellar astrocytoma, malignant glioma (e.g. cerebral astrocytoma), cervical cancer, chronic lymphocytic leukemia, chronic myelogenous leukemia, colon cancer, cutaneous T-cell lymphoma, desmoplastic small round cell tumor, endometrial cancer, ependymoma, esophageal cancer, Ewing's sarcoma, extracranial germ cell tumor (e.g. childhood), extragonadal germ cell tumor, extrahepatic bile duct cancer, eye cancer (e.g. intraocular melanoma, retinoblastoma), gallbladder cancer, gastric (stomach) cancer, gastrointestinal stromal tumor, germ cell tumor (e.g. childhood extracranial), gestational trophoblastic tumor, hairy cell leukemia, head and neck cancer, hepatocellular (liver) cancer, Hodgkin lymphoma, other lymphomas (AIDS-related, non-Hodgkin, primary central nervous system), hypopharyngeal cancer, intraocular melanoma, islet cell carcinoma, Kaposi sarcoma, laryngeal cancer, leukemias (e.g. acute lymphoblastic, chronic lymphocytic, chronic myelogenous, hairy cell), lip and oral cavity cancer, primary liver cancer, small cell lung cancers, non-small cell lung cancer, macroglobulinemia (Waldenstrom), malignant fibrous histiocytoma of bone, medulloblastoma (e.g. childhood), intraocular melanoma, other melanomas, Merkel cell carcinoma, mesotheliomas (e.g. adult malignant, childhood), metastatic squamous neck cancer with occult primary, mouth cancer, multiple endocrine neoplasia syndrome (e.g. in a pediatric patient), plasma cell neoplasms (e.g. multiple myeloma), mycosis fungoides, myelogenous leukemia (e.g. chronic), nasal cavity and paranasal sinus cancer, nasopharyngeal carcinoma, neuroblastoma, oral cancer, oropharyngeal cancer, osteosarcoma, ovarian cancer, ovarian epithelial cancer (e.g. surface epithelial-stromal tumor), ovarian germ cell tumor, ovarian low malignant potential tumor, islet cell pancreatic cancer, other pancreatic cancers, paranasal sinus and nasal cavity cancer, parathyroid cancer, penile cancer, pharyngeal cancer, pheochromocytoma, pineal astrocytoma, pineal germinoma, pincoblastoma and supratentorial primitive neuroectodermal tumors (childhood), pituitary adenoma, plasma cell neoplasia, pleuropulmonary blastoma, primary central nervous system lymphoma, prostate cancer, rectal cancer, renal cell carcinoma (kidney cancer), renal pelvis and ureter transitional cell cancer, retinoblastoma, rhabdomyosarcoma (childhood), salivary gland cancer, soft tissue sarcoma, uterine sarcoma, Sezary syndrome, melanoma, skin carcinoma (e.g. Merkel cell), other skin cancers, small intestine cancer, squamous cell carcinoma, supratentorial primitive neuroectodermal tumor (e.g. childhood), testicular cancer, throat cancer, thymoma (e.g. childhood), thymic carcinoma, thyroid cancer (childhood or adult), urethral cancer, endometrial uterine cancer, vaginal cancer, vulvar cancer, Waldenstrom macroglobulinemia, and Wilms tumor.

As mentioned, in certain embodiments, the iodine-releasing compound is administered with an additional therapeutic agent. In certain embodiments, the additional agent a different iodine-containing compound. In certain embodiments, the two or more iodine-containing compounds form an equilibrium. Non-limiting examples of such combinations of iodine-containing compounds are molecular iodine combined with an iodide compound, e.g., sodium iodide, potassium iodide, or hydrogen iodide. In certain embodiments, the additional agent is a chemotherapy agent. In more specific embodiments, the chemotherapy agent may be selected from alkylating and alkylating-like agents such as nitrogen mustards (e.g., chlorambucil, chlormethine, cyclophosphamide, ifosfamide, and melphalan), nitrosoureas (e.g., carmustine, fotemustine, lomustine, and streptozocin), platinum agents (e.g., alkylating-like agents) (e.g., carboplatin, cisplatin, oxaliplatin, BBR3464, and satraplatin), busulfan, dacarbazine, procarbazine, temozolomide, thioTEPA, treosulfan, and uramustine; antimetabolites such as folic acids (e.g., aminopterin, methotrexate, pemetrexed, and raltitrexed); purines such as cladribine, clofarabine, fludarabine, mercaptopurine, pentostatin, and thioguanine; pyrimidines such as capecitabine, cytarabine, fluorouracil, floxuridine, and gemcitabine; spindle poisons/mitotic inhibitors such as taxanes (e.g., docetaxel, paclitaxel, cabazitaxel) and vincas (e.g., vinblastine, vincristine, vindesine, and vinorelbine); cytotoxic/antitumor antibiotics such anthracyclines (e.g., daunorubicin, doxorubicin, epirubicin, idarubicin, mitoxantrone, pixantrone, and valrubicin), compounds naturally produced by various species of Streptomyces (e.g., actinomycin, bleomycin, mitomycin, plicamycin) and hydroxyurea; topoisomerase inhibitors such as camptotheca (e.g., camptothecin, topotecan and irinotecan) and podophyllums (e.g., etoposide, teniposide); monoclonal antibodies for cancer immunotherapy such as anti-receptor tyrosine kinases (e.g., cetuximab, panitumumab, trastuzumab), anti-CD20 (e.g., rituximab and tositumomab), and others for example alemtuzumab, bevacizumab, and gemtuzumab; photosensitizers such as aminolevulinic acid, methyl aminolevulinate, porfimer sodium, and verteporfin; tyrosine kinase inhibitors such as cediranib, dasatinib, erlotinib, gefitinib, imatinib, lapatinib, nilotinib, sorafenib, sunitinib, and vandetanib; serine/threonine kinase inhibitors, (e.g., inhibitors of AbI, c-Kit, insulin receptor family member(s), EGF receptor family member(s), Akt, mTOR [e.g., rapamycin or analogs thereof, direct inhibitors of mTORC1 and/or mTORC2], Raf kinase family, phosphatidyl inositol (PI) kinases such as PI3 kinase, PI kinase-like kinase family members, cyclin dependent kinase family members, and aurora kinase family), growth factor receptor antagonists, retinoids (e.g., alitretinoin and tretinoin), altretamine, amsacrine, anagrelide, arsenic trioxide, asparaginase (e.g., pegaspargase), bexarotene, bortezomib, denileukin diftitox, estramustine, ixabepilone, masoprocol, mitotane, and testolactone, Hsp90 inhibitors, proteasome inhibitors, HDAC inhibitors, angiogenesis inhibitors, e.g., anti-vascular endothelial growth factor agents such as bevacizumab or VEGF-Trap, matrix metalloproteinase inhibitors, and pro-apoptotic agents (e.g., apoptosis inducers). In other embodiments, the additional therapeutic agent has activity against triple-negative breast cancer. Non-limiting examples of such agents are anthracycline; paclitaxel; docetaxel; eribulin; ixabepilone; capecitabine; Tigatuzumab; 3-(phenylethynyl)-1H-pyrazolo[3,4-d]pyrimidin-4-amine derivatives; Teriflunomide; carboplatin, CB(2) cannabinoid o-quinone compounds; alantolactone; cabazitaxel; and dutasteride.

In other embodiments, there is provided a pharmaceutical composition, comprising an iodine-containing compound and an ion channel. The iodine-containing compound may be any compound or formulation mentioned herein, each of which represents a separate embodiment. The ion channel may be any channel mentioned herein, each of which represents a separate embodiment.

In yet other embodiments, there is provided a method for treating a subject infected with an intracellular pathogen, comprising administering a pharmaceutical composition, comprising an ion channel and an iodine-containing compound.

In still other embodiments, there is provided a method for treating a subject infected with an intracellular pathogen, comprising: (a) administering a pharmaceutical composition comprising an ion channel; and (b) subsequent to step (a) administering a pharmaceutical composition comprising an iodine-containing compound.

In other embodiments, there is provided a method for treating a neoplasm in a subject in need thereof, comprising administering a pharmaceutical composition, comprising an ion channel and an iodine-containing compound. In yet other embodiments, there is provide a method for treating a neoplasm in a subject in need thereof, comprising: (a) administering a pharmaceutical composition comprising an ion channel; and (b) subsequent to step (a) administering a pharmaceutical composition comprising an iodine-containing compound.

In yet other embodiments, there is provided a pharmaceutical composition, comprising a polyphenol and an iodine-containing compound. The iodine-containing compound may be any compound or formulation mentioned herein, each of which represents a separate embodiment. In more specific embodiments, the polyphenol is selected from resveratrol, quercetin, kaempferol, secoisolariciresinol diglycoside (SDG), a glucosinolate (Drewnowski A et al.), and a catechin (non-limiting examples of which are epicatechin (EC), epicatechin-3-gallate (ECG), epigallocatechin (EGC) and epigallocatechin-3-gallate (EGCG)). Therapeutic use of polyphenols is known in the art, and is described, for example, in Niedzwiecki A et al., and the references cited therein.

Those skilled in the art will appreciate that polyphenols are categorized into phenolic acids, stilbenes (e.g., resveratrol [Hosseinimehr et al.]), lignans (e.g., secoisolariciresinol diglycoside [SDG]), and flavonoids. Flavonoids in turn are subcategorized into flavonols (e.g., quercetin and kaempferol), flavones, isoflavones, flavanones, anthocyanidins, and flavanols (catechins and proanthocyanidins)

In other embodiments, there is provided a method for treating a subject infected with an intracellular pathogen, comprising administering a pharmaceutical composition, the pharmaceutical composition comprising a polyphenol and an iodine-containing compound. In yet other embodiments, there is provided a method for treating a subject infected with an intracellular pathogen, comprising: (a) administering a pharmaceutical composition comprising a polyphenol; and (b) subsequent to step (a), administering a pharmaceutical composition comprising an iodine-containing compound.

In still other embodiments, there is provided a method for treating a neoplasm in a subject in need thereof, comprising administering a pharmaceutical composition, comprising a polyphenol and an iodine-containing compound, thereby treating a neoplasm. In other embodiments, there is provided a method for treating a neoplasm in a subject in need thereof, comprising: (a) administering a pharmaceutical composition comprising a polyphenol; and (b) subsequent to step (a), administering a pharmaceutical composition comprising an iodine-containing compound.

In various embodiments where an ion channel and an iodine-containing compound, or in other embodiments a polyphenol and an iodine-containing compound are sequentially administered, an iodine-containing compound is administered, in some embodiments, within 24 hours of administration of the ion channel or polyphenol. In other embodiments, the iodine-containing compound is administered within 48, 36, 20, 18, 15, 12, 10, 8, 6, 5, 4, 3, 2, or 1 hours of administration of the ion channel or polyphenol. In certain embodiments, if multiple doses of the iodine-containing compound are administered, the first dose thereof is administered within any of the specified timeframes of administration of the ion channel or polyphenol, each of which represents a separate embodiment. In yet other embodiments, if multiple doses of the therapeutic combination are administered, the therapeutic combination is indicated for administration of each dose of the iodine-containing compound within any of the specified timeframes of administration of the ion channel or polyphenol, each of which represents a separate embodiment.

### Targeting Moieties

The described targeting moiety is, in certain embodiments, a receptor of a virus, non-limiting examples of which are sialylated glycans, integrins, and phosphatidylserine receptors. In certain embodiments, the described liposomes or nanoparticles display viral receptors. In more specific embodiments, such compositions can neutralize and/or target extracellular viruses. In another embodiment, this is administered in conjunction with another form of iodine delivery.

In the case of cancers, the targeting moiety, is, in some embodiments, a ligand of a surface protein or other surface molecule of the cancer cell, or an antibody to a surface protein or molecule. Non-limiting examples of such moieties are antibodies to angiogenic growth factors, such as vascular endothelial growth factor (VEGF), angiopoietin 1 (ANG1), and basic fibroblast growth factor (bFGF) (e.g., for breast and colon cancers); estrogen receptor or another breast cancer marker; folate; transferrin; and luteinizing hormone (e.g., for breast, ovarian, and prostate tumors). Methods of conjugating liposomes to antibodies are known in the art. Non-limiting examples include Avidin-conjugated antibodies and Biotinylated liposomes, reagents for which are available from Encapsula Nanosciences (Brentwood, TN).

In other embodiments, the targeting moiety is an antibody to a class I major histocompatibility complex protein or other marker indicating that the cell is infected. In other embodiments, the moiety is an antibody to a marker of a malignant, transformed, or cancer cell.

### EXPERIMENTAL DETAILS SECTION

### Example 1: Treatment of Breast Cancer with Conjugated Anti-Cea Antibodies

Anti-CEA antibodies are conjugated to non-radioactive iodide by the n-bromosuccinimide method (Adam et al.) as described in Lane et al., albeit with non-radioactive iodide. The conjugated antibodies are used to treat CEA-expressing breast cancer tumors in a mouse model, for example as described in Chan et al.

### Example 2: Treatment of Tuberculosis (Tb) with Liposomes Conjugated to Anti-Tb Antibodies

Non-radioactive iodide is incorporated into liposomes as described in Homann et al. the liposomes are conjugated to antibodies against tuberculosis (TB) antigens such as 19 kD lipoprotein (Noss EH, et al.). The conjugated antibodies are used to treat TB infection in a mouse model, for example as described in Silvério, et al.

### REFERENCES

Adam et al., Radioiodination for Therapy. Ann Clin Biochem. 26(3): 244-245.

Benke et al., Iodide-Selective Synthetic Ion Channels Based on Shape-Persistent Organic Cages. J Am Chem Soc 139(22):7432-35.

Bigliardi et al., Povidone iodine in wound healing: A review of current concepts and practices, Int. J. Surgery 44:260-268.

Chan et al. Novel mouse model for carcinoembryonic antigen-based therapy. Molecular Therapy Volume 9, Issue 6, June 2004, Pages 775-785.

Drewnowski A., Gomez-Carneros C. Bitter taste, phytonutrients and the consumer: A review. Am. J. Clin. Nutr. 2000;72:1424-1435.

Gorbalenya, AE et al. The species Severe acute respiratory syndrome-related coronavirus: classifying 2019-nCOV and naming it SARS-COV-2. Nat Microbiol (2020).

Homann et al., A liposome hydrogel with polyvinyl-pyrrolidone iodine in the local treatment of partial-thickness burn wounds. Ann Plast Surg. 2007 October;59 (4):423-7.

Hosseinimehr et al., Resveratrol Sensitizes Selectively Thyroid Cancer Cell to 131-Iodine Toxicity, Journal of Toxicology, Volume 2014, Article ID 839597.

Goldman et al. 2017, Nanoparticles target early-stage breast cancer metastasis in vivo, Nanotechnology 28 43LT01.

Lane et al., Radioimmunotherapy of metastatic colorectal tumours with iodine-131-labelled antibody to carcinoembryonic antigen: phase I/II study with comparative biodistribution of intact and F(ab′)2 antibodies. Br J Cancer 1994 September;70(3):521-5.

Li et al. Analysis of the Antigenic Properties of Membrane Proteins of Mycobacterium tuberculosis. Scientific Reports volume 9, Article number: 3042 (2019).

Liu et al., Engineered Ionic Gates for Ion Conduction Based on Sodium and Potassium Activated Nanochannels. J. Am. Chem. Soc. 2015, 137, 37, 11976-11983.

Micali et al. Sodium iodide symporter (NIS) in extrathyroidal malignancies: focus on breast and urological cancer. BMC Cancer 2014, 14:303 (2014),

Niedzwiecki A et al., Anticancer Efficacy of Polyphenols and Their Combinations. Nutrients. 2016 September; 8(9):552.

Noss EH, et al. Toll-like receptor 2-dependent inhibition of macrophage class II MHC expression and antigen processing by 19 kD lipoprotein of Mycobacterium tuberculosis. J. Immunol. 2001;167:910-918.

How Molecular Iodine Attacks Breast Cancer, Oncology Times: (2016)-38(24):34)

PhysOrg 2017. Synthetic nanochannels for iodide transport (2017 June 8) retrieved from https://phys.org/news/2017-06-synthetic-nanochannels-iodide.html.

Reimer et al. Povidone-iodine liposomes—an overview. Dermatology. 1997;195 Suppl 2:93-9.

Roy et al., Polyhydrazide-Based Organic Nanotubes as Efficient and Selective Artificial Iodide Channels. Angewandte Chemie 59(12):4806-4813 (2020).

Siddell, SG et al., (Additional changes to taxonomy ratified in a special vote by the International Committee on Taxonomy of Viruses (October 2018). Arch. Virol. 164, 943-946 [2019]);

Silvério, et al. Advances on the Role and Applications of Interleukin-1 in Tuberculosis. mBio. 2021 Nov-Dec; 12(6): e03134-21.

Son et al., A novel strategy of transferring NIS protein to cells using extracellular vesicles leads to increase in iodine uptake and cytotoxicity. Int J Nanomedicine 2019, 14:1779-1787.

Sutradhar et al., Nanotechnology in Cancer Drug Delivery and Selective Targeting. ISRN Nanotechnology Volume 2014, Article ID 939378.

Wu et al., Prospects and Challenges in Anion Recognition and Transport. Chem (Cellpress) 6:1296-1309.

Ziebuhr, J et al. (Proposal 2017.013S. A.v1. Reorganization of the family Coronaviridae into two families, Coronaviridae (including the current subfamily Coronavirinae and the new subfamily Letovirinae) and the new family Tobaniviridae (accommodating the current subfamily Torovirinae and three other subfamilies), revision of the genus rank structure and introduction of a new subgenus rank. (ICTV, 2017); https://ictv.global/proposal/2017.Nidovirales/.);

Ziebuhr, J. et al. (Proposal 2019.021S.Ac.v1. Create ten new species and a new genus in the subfamily Orthocoronavirinae of the family Coronaviridae and five new species and a new genus in the subfamily Serpentovirinae of the family Tobaniviridae. (ICTV, 2019); https://ictv.global/proposal/2019.Nidovirales/);

1. A pharmaceutical therapeutic composition for internal use, comprising a non-radioactive iodine releasing compound conjugated to a carrier, wherein said carrier is an antibody.

2. The pharmaceutical composition of claim 1 for treating a neoplasm.

3. (canceled)

4. The pharmaceutical composition of claim 1 for treating an intracellular pathogen.

5-11. (canceled)

12. The pharmaceutical composition of claim 1, further comprising an additional iodine-containing compound that forms an equilibrium with said iodine-releasing compound.

13-19. (canceled)

20. The pharmaceutical composition of claim 1, wherein the composition additionally comprises a polyphenol.

21. The pharmaceutical composition of claim 20, wherein said polyphenol is selected from the group consisting of resveratrol, quercetin, kaempferol, secoisolariciresinol diglycoside (SDG), a glucosinolate, epicatechin (EC), epicatechin-3-gallate (ECG), epigallocatechin (EGC) and epigallocatechin-3-gallate (EGCG).

22. The pharmaceutical composition of claim 1 for use in treating a subject infected with an intracellular pathogen, wherein:
a. a pharmaceutical composition comprising a polyphenol is administered; and
b. subsequent to step (a) said pharmaceutical composition is administered,
thereby treating a subject infected with an intracellular pathogen.

23. The pharmaceutical composition of claim 1 for use in treating a neoplasm in a subject in need thereof, wherein:
a. a pharmaceutical composition comprising a polyphenol is administered; and
b. subsequent to step (a) said pharmaceutical composition is administered, thereby treating a neoplasm.

24. The pharmaceutical therapeutic composition of claim 1 wherein the antibody is conjugated with a nanoparticle, a nanosphere, a nanocapsule, nanotube, a polymeric micelle or a liposome.

25. The pharmaceutical composition of claim 24 for treating a neoplasm.

26. The pharmaceutical composition of claim 24 for treating an intracellular pathogen.

27. The pharmaceutical composition of any of claims 24, wherein said iodine-releasing compound is disposed within said nanoparticle, polymeric micelle, nanosphere, nanocapsule, or nanotube.

28. The pharmaceutical composition of claim 24, wherein said nanoparticle, polymeric micelle, nanosphere, nanocapsule, or nanotube comprises a targeting moiety.

29. The pharmaceutical composition of claim 24, wherein said nanoparticle is a liposome.
